SEQUENCE LISTING (2 sequences)

SEQ ID NO: 1 - RNA, 24 nt, Hordeum vulgare
  misc_feature: RNA sequence of hvu-miR168-3p
gaucccgccuugcaccaagugaau

SEQ ID NO: 2 - RNA, 21 nt, Aegilops tauschii
  misc_feature: RNA sequence of ata-miR168-5p
ucgcuuggugcagaucgggac

(19) United States
(12) Patent Application Publication
(10) Pub. No.: US 2024/0263174 A1
(43) Pub. Date: Aug. 8, 2024

(54) EXPRESSION-INDUCING AGENT FOR CLASS I GLUCOSE TRANSPORTER (GLUT), AND MEDICINAL COMPOSITION AND FOOD COMPOSITION USING SAME

(71) Applicant: NATIONAL UNIVERSITY CORPORATION TOKAI NATIONAL HIGHER EDUCATION AND RESEARCH SYSTEM, Aichi (JP)

(72) Inventors: Yukihiro Akao, Aichi (JP); Hironobu Murase, Aichi (JP)

(73) Assignee: NATIONAL UNIVERSITY CORPORATION TOKAI NATIONAL HIGHER EDUCATION AND RESEARCH SYSTEM, Aichi (JP)

(21) Appl. No.: 17/284,597

(22) PCT Filed: Oct. 11, 2019
(86) PCT No.: PCT/JP2019/040270

§ 371 Date: Jul. 12, 2021

(30) Foreign Application Priority Data
Oct. 12, 2018 (JP) 2018-193615

(51) Int. Cl. C12N 15/113

(52) U.S. Cl. CPC C12N 15/113; C12N 2310/141

(57) ABSTRACT

To provide a strong candidate for prevention or treatment of a disease or disorder related to glucose metabolism, by discovering a substance capable of inducing the expression of the GLUT family. The followings are provided by some aspects of the present invention: an expression inducer of class I glucose transporter (GLUT) comprising, as an active ingredient, at least one oligonucleotide selected from the group consisting of hvu-miR168-3p and ata-miR168-5p, their precursors and their derivatives; a pharmaceutical composition and a food or beverage composition containing the expression inducer (for example, a composition for prevention or treatment (improvement) of a disease or disorder related to glucose metabolism).

### TECHNICAL FIELD

The present invention relates to an expression inducer of class I glucose transporter (GLUT), and a pharmaceutical composition and a food and beverage composition using the same.

### BACKGROUND ART

Glucose is an essential molecule in the living body. In order for this glucose to pass through a biological membrane, a membrane protein called a glucose transporter (glucose transporter: also referred to as “GLUT” in the present specification) is required on a cell membrane. Several classes and molecules have been identified as GLUT, but expression sites and roles of GLUT have been relatively well elucidated in four types of molecules GLUT1 to GLUT4 belonging to class I, their molecular weights are all about 50 kDa, and GLUT1 to GLUT4 have a common structure that penetrates the cell membrane twelve times.

On the other hand, GLUT has different tissue distribution, intracellular distribution and affinity for sugar for each isoform, and each has its own characteristics. Among them, GLUT-1 has a high affinity (Kₘ: 1 to 5 mM) for glucose, and its distribution in humans have been reported in erythrocyte, fetal tissue, brain, kidney, cancerous tissue, and the like. In addition, GLUT-3 also has a high affinity (Kₘ: 1 to 5 mM) for glucose, and distribution of GLUT-3 in humans have been reported in brain, placenta, kidney, liver, adipose tissue, small intestine, and the like.

Further, GLUT4 is found in adipose tissue and striated muscles such as skeletal muscle. While GLUT4 is normally present inside cells, it is insulin-sensitive and transfers (translocates) onto the cell membrane in response to insulin stimulation, and has a function of transporting glucose from the blood to tissues and lowering blood sugar levels.

In addition, GLUT2 is expressed in renal tubular epithelial cells, small intestinal epithelial cells, hepatocytes, and β cells of pancreas. GLUT2 present in the β cells of pancreas is involved in insulin secretion, and has a mechanism that, when the blood sugar level rises due to diet or the like, glucose is taken up into the β cells of pancreas via GLUT2 in a membrane of the B cells, and insulin is secreted by the stimulation. Furthermore, in the liver, which is another expression site, GLUT2 is present in a cell membrane from the beginning before stimulation with insulin, and other GLUT is substantially absent. Therefore, it is said that uptake of glucose into cells in the liver does not depend on the action of insulin.

Here, a substance capable of inducing the expression of the GLUT family is considered to be a strong candidate for prevention or treatment of a disease or disorder related to glucose metabolism.

Incidentally, it is known that miRNA (microRNA) has various physiological activities. miRNA is a cellular endogenous non-coding RNA of about 20 to 25 bases. miRNA is first transcribed as a primary transcript (pri-miRNA) with a length of about several hundred to several thousand bases, from miRNA gene on genomic DNA. Subsequently, the primary transcript is processed to become pre-miRNA having a hairpin structure of about 60 to 70 bases. Thereafter, the pre-miRNA moves from a nucleus into cytoplasm and is further processed to become mature miRNA consisting of a dimer (guide strand and passenger strand) of about 20 to 25 bases. It is known that mature miRNA functions to inhibit translation of a target gene as the guide strand (antisense strand) in the mature miRNA forms a complex with a protein called RISC (RNA-Induced Silencing Complex) and acts on the mRNA of the target gene.

Conventionally, many families with various origins are known as miR-168, which is one of miRNAs (Non-Patent Literature 1), and for example, hvu-miR168-3p originating from barley (Hordeum vulgare), ata-miR168-5p originating from wheat (Aegilops tauschii) and the like are known. However, nothing is known about an effect of these miR-168 families on the expression of the GLUT family.

### CITATION LIST

### Non-Patent Literature

Non-Patent Literature 1: “miRbase”, [online], [Searched Jul. 30, 2018], Internet <http://www.mirbase.org/>

### SUMMARY OF INVENTION

### Technical Problem

An object of the present invention is to provide a strong candidate for prevention or treatment of a disease or disorder related to glucose metabolism, by discovering a substance capable of inducing the expression of the GLUT family.

### Solution to Problem

The present inventor has conducted intensive investigations in consideration of the above problem. As a result, surprisingly, hvu-miR168-3p originating from barley (Hordeum vulgare) and ata-miR168-5p originating from wheat (Aegilops tauschii) were found to induce the expression of class I GLUT. Based on this finding, the present inventor has completed the present invention as a use of the above two types of miR-168 families.

That is, according to one aspect of the present invention, an expression inducer of class I glucose transporter (GLUT) including, as an active ingredient, at least one oligonucleotide selected from the group consisting of hvu-miR168-3p and ata-miR168-5p, their precursors and their derivatives is provided.

According to the present invention, a strong candidate for prevention or treatment of a disease or disorder related to glucose metabolism may be provided.

### BRIEF DESCRIPTION OF DRAWINGS

FIG. 1 is a photograph showing results of measuring expression levels of GLUT1 protein by Western blotting for human skin-derived fibroblasts transfected with hvu-miR168-3p or ata-miR168-5p in Example (Test Example 1).

FIG. 2 is a graph showing results of measuring viable cell counts by trypan blue staining for human skin-derived fibroblasts transfected with hvu-miR168-3p having different concentrations after the cells were cultured under different nutritional conditions in Example (Test Example 2).

FIG. 3 is a graph showing results of measuring blood glucose concentration (blood sugar levels) of nude mice (ICR mice), to which hvu-miR168-3p was administered, after performing a sugar load test on the mice in Example (Test Example 3).

FIG. 4 is a photograph showing results of measuring expression levels of GLUT1 protein by Western blotting for brain tissue homogenates of nude mice (ICR mice), to which hvu-miR168-3p was administered, in Example (Test Example 3).

FIG. 5 is a photograph showing results of measuring expression levels of GLUT1 protein by Western blotting for human rhabdomyosarcoma-derived cells transfected with hvu-miR168-3p or ata-miR168-5p in Example (Test Example 4).

FIG. 6 is a photograph showing results of measuring expression levels of GLUT1 protein by Western blotting for human rhabdomyosarcoma-derived cells transfected with hvu-miR168-3p or ata-miR168-5p after the cells were cultured in a medium supplemented with 5% (v/v) FCS in Example (Test Example 4).

FIG. 7 is a graph showing results of measuring viable cell counts by measuring absorbance for human rhabdomyosarcoma-derived cells transfected with hvu-miR168-3p or ata-miR168-5p having different concentrations after the cells were cultured under different nutritional conditions in Example (Test Example 5).

FIG. 8 is a graph showing results of measuring intracellular ATP levels of human rhabdomyosarcoma-derived cells (cells cultured in a medium supplemented with 5% (v/v) FCS) transfected with different concentrations of hvu-miR168-3p or ata-miR168-5p in Example (Test Example 5).

FIG. 9 is a graph showing results of measuring changes in mRNA expression levels of Ndufs6 (NADH dehydrogenase [ubiquinone] iron-sulfur protein 6, mitochondrial), which is a gene involved in oxidative phosphorylation pathway (OXPHOS) in mitochondrial complex I, in ASF-4-1 cells and JCRB9072 cells, into which hvu-miR168-3p was introduced, by real-time PCR method, in Example (Test Example 8).

### DESCRIPTION OF EMBODIMENTS

Hereinafter, embodiments of the present invention will be described in detail.

### <<Oligonucleotide>>

### [Basic Structure]

Oligonucleotides disclosed herein are at least one selected from the group consisting of hvu-miR168-3p and ata-miR168-5p (these are defined as “mature miRNAs”), their precursors and their derivatives. Here, from the viewpoint of being superior in an expression-inducing effect of class I GLUT, at least one oligonucleotide selected from the group consisting of hvu-miR168-3p, its precursors and their derivatives is preferably used. Meanwhile, in the present specification, the “precursor” is a concept that includes all precursors including pri-miRNA and pre-miRNA before becoming mature miRNA. Also, in the present specification, the “derivative” is a concept that includes all modified miRNAs obtained when mature miRNA receives any modification.

Here, the “hvu-miR168” is a microRNA originating from barley (Hordeum vulgare), and its stem-loop structure is as follows.

```
[Chemical 1]
    guc c c c g gc au ccc - cc
5′ cgu gc gccg cuc g ucgcuuggugcag cggga uccg cccg c
          ||| || |||| ||| | ||||||||||||| ||||| |||| ||||
3′ gcg cg cggc gag c agugaaccacguu gcccu aggc gggc c
    --- a - c g ua cc --- c ag
```

Then, the “hvu-miR168-3p” is a single-stranded RNA expressed from a strand on 3′-side of hvu-miR168, to which accession number MIMAT0018216 of miRbase is assigned. That is, the “hvu-miR168-3p” has a base sequence represented by SEQ ID NO: 1 below.

```
[Chemical 2]
(1) SEQ ID NO: 1:
5′-gaucccgccuugcaccaagugaau-3′;
[Chemical 3]
    -- c g gc au c uca cc
5′ cg cuc g ucgcuuggugcag cggga cc gcccg c
        || ||| | ||||||||||||| ||||| || ||||| |
3′ gc gag c agugaaccacguu gcccu gg cgggc c
    cg c g ua cc a --c ag
```

Moreover, the “ata-miR168-5p” is a single-stranded RNA expressed from a strand on 5′-side of ata-miR168, to which accession number MI0031679 of miRbase is assigned. That is, the “ata-miR168-5p” has a base sequence represented by SEQ ID NO: 2 below.

[Chemical 4]
(2) SEQ ID NO: 2:
5′-ucgcuuggugcagaucgggac-3′

### [Derivatives]

The active ingredient of the agent according to the present invention may be a derivative in which an oligonucleotide having the above-mentioned basic structure has been chemically modified by means that are known to those ordinarily skill in the art, for the purpose of incorporation into cells, enhancement of resistance to RNase, and the like. Examples of such chemical modification include: (a) addition of a chemically modifying group to 3′-terminal of an oligonucleotide chain, (b) substitution of a constituent base by a base containing a modified sugar moiety, (c) substitution of a phosphodiester bond by a phosphorus atom-modified bond, and the like.

### (a) Addition of Chemically Modifying Group to 3′-Terminal

Among them, the oligonucleotide chain that has been subjected to the chemical modification of (a) (addition of a chemically modifying group to 3′-terminal) is, for example, one having a 3′-terminal modified structure represented by the following chemical formula 1, in an oxygen atom of a phosphodiester bond on the 3′-terminal side bonded to a base of a nucleotide at the 3′-terminal of the above-mentioned oligonucleotide chain.

[Chemical 5]

-L-M-3′ [Chemical Formula 1]

Here, in the chemical formula (1), L represents a substituted or unsubstituted cyclic compound-containing group represented by any of the following chemical formulas (2a) to (2g):

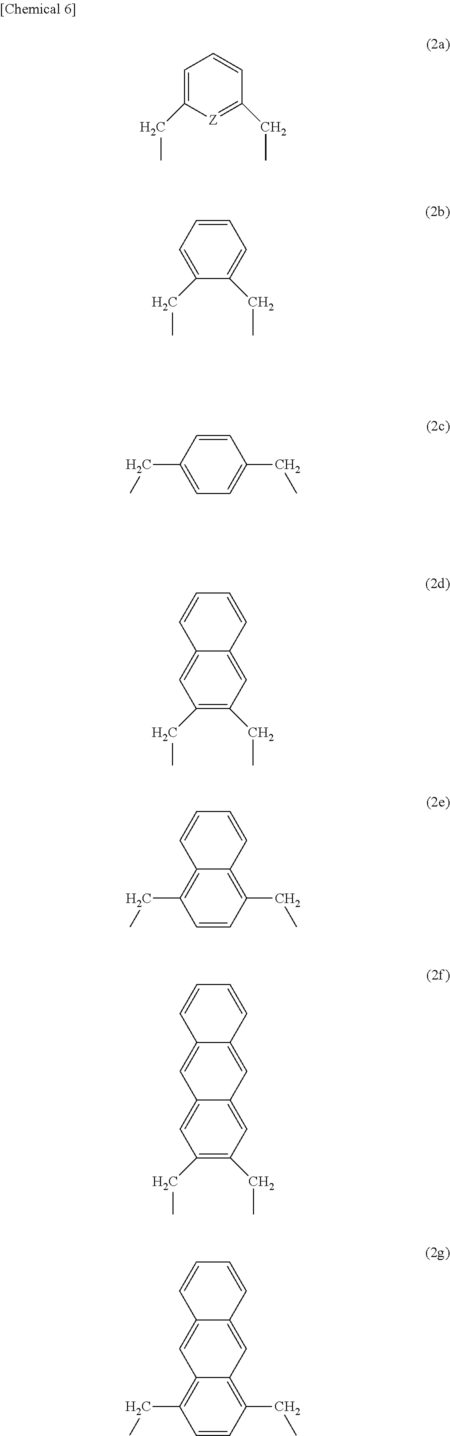

or represents a divalent group in which two or more of the cyclic compound-containing groups are respectively linked through a phosphodiester bond; and M represents a hydrogen atom or a hydroxyl protecting group.

In a preferred embodiment, the L represents a divalent group in which two or more (for example, 2 to 10, preferably 2 to 6, more preferably 2 to 4, further preferably 2 to 3, and particularly preferably 2) of the cyclic compound-containing groups are respectively linked through a phosphodiester bond.

Here, in a case where the cyclic compound-containing group is substituted, examples of the substituent that substitutes the cyclic compound-containing group include halogen such as fluorine, chlorine, bromine, and iodine, alkyl groups such as a methyl group, an ethyl group, a tert-butyl group, and a dodecyl group; aryl groups such as a phenyl group, a p-tolyl group, a xylyl group, a cumenyl group, a naphthyl group, an anthryl group, and a phenanthryl group; alkoxy groups such as a methoxy group, an ethoxy group, and a tert-butoxy group; aryloxy groups such as a phenoxy group and a p-tolyloxy group; alkoxycarbonyl groups such as a methoxycarbonyl group, a butoxycarbonyl group, a 2-ethylhexyloxycarbonyl group, and a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; alkylsulfanyl groups such as a methylsulfanyl group and a tert-butylsulfanyl group; arylsulfanyl groups such as a phenylsulfanyl group and a p-tolylsulfanyl group; alkylamino groups such as a methylamino group and a cyclohexylamino group, dialkylamino groups such as a dimethylamino group, a diethylamino group, a morpholino group, and a piperidino group; arylamino groups such as a phenylamino group, a p-tolylamino group, and the like; as well as a hydroxy group, a carboxy group, a formyl group, a mercapto group, a sulfo group, a mesyl group, a p-toluenesulfonyl group, an amino group, a nitro group, a cyano group, a trifluoromethyl group, a trichloromethyl group, a trimethylsilyl group, a phosphinico group, a phosphono group, and the like.

Further, in the chemical formula (2a), Z represents CH or N. In a preferred embodiment, the L includes one or two or more cyclic compound-containing groups represented by the chemical formula (2a), which may be substituted. Moreover, in another preferred embodiment, the L represents a divalent group in which two or more (for example, 2 to 10, preferably 2 to 6, more preferably 2 to 4, further preferably 2 to 3, and particularly preferably 2) of the cyclic compound-containing groups represented by the chemical formula (2a) are respectively linked through a phosphodiester bond. At this time, L is preferably a divalent group that includes, among the cyclic compound-containing groups represented by the chemical formula (2a), both a group in which Z is CH (that is, a group containing a benzene ring) and a group in which Z is N (that is, a group containing a pyridine ring), and more preferably a divalent group formed from one each of these groups. Also, the L has a structure represented by the following chemical formula (3):

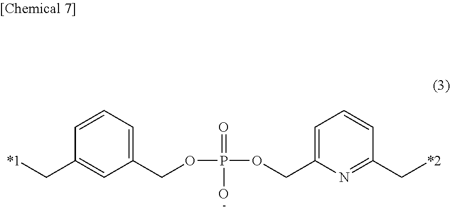

in a further preferred embodiment.

Here, in the chemical formula (3), *1 represents a bonding site to the oxygen atom of a phosphodiester bond on the 3′-terminal side bonded to the base of a nucleotide at the 3′-terminal of the oligonucleotide chain; and *2 represents a bonding site to M.

In the chemical formula (1), M represents a hydrogen atom or a hydroxyl protecting group, and preferably represents a hydrogen atom. Therefore, in the most preferred embodiment, the structure represented by “-L-M” is a group in which a hydrogen atom is bonded to *2 of the chemical formula (3). Meanwhile, the hydroxyl protecting group may be a group that protects oxygen in a hydroxyl group that is substituted by the protecting group from an unintended reaction, and conventionally known findings can be referred to as appropriate. Preferably, the hydroxyl protecting group is one that can be removed with maintaining the activity of an oligonucleotide derivative. Such a hydroxyl protecting group is not particularly limited, and is, for example, a fluorenylmethoxycarbonyl (FMOC) group, a dimethoxytrityl (DMT) group, a monomethoxytrityl group, a trifluoroacetyl group, a levulinyl group, or a silyl group.

### (b) Substitution of Constituent Base by a Base Containing a Modified Sugar Moiety

In a case where the oligonucleotide chain is subjected to the chemical modification of (b) (substitution of a constituent base by a base containing a modified sugar moiety), the nucleotides that constitute the oligonucleotide chain can be each independently substituted by a base containing a modified sugar moiety selected from the group consisting of 2′-O-methyl, 2′-methoxyethoxy, 2′-fluoro, 2′-allyl, 2′-O-[2(methylamino)-2-oxoethyl], 4′-thio, 4′-(CH₂)₂—O-2′-crosslinking, 2′-locked nucleic acid, or 2′-O—(N-methylcarbamate). Among them, it is preferable that the modified sugar moiety includes 2′-O-methyl or 2′-fluoro modification.

### (c) Substitution of a Phosphodiester Bond by a Phosphorus Atom-Modified Bond

The phosphodiester bonds that constitute the oligonucleotide chain can be each independently substituted by a phosphorus atom-modified bond represented by the following chemical formula (4):

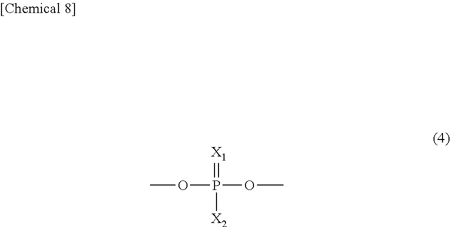

in a case where the oligonucleotide chain is subjected to the chemical modification of (c) (substitution of a phosphodiester bond by a phosphorus atom-modified bond).

In the chemical formula (4), X¹ independently represents O, S or Se, and X² independently represents OH or O⁻, SH or S⁻, SeH or Se⁻, an alkyl group having 1 to 4 carbon atoms or a morpholino group. However, in a case where X¹ represents O and X² represents O⁻, the chemical formula (4) represents a normal phosphodiester bond. Therefore, such a case is not to be included in the scope of the present invention. In a preferred embodiment, X¹ is O, and X² represents SH or S⁻, SeH or Se⁻, an alkyl having 1 to 4 carbon atoms or a morpholino group. Also, in a more preferred embodiment, X¹ is O, and X² represents SH or S⁻. Further, in a particularly preferred embodiment, X¹ is O, and X² represents SH or S⁻ (in this case, the phosphorus atom-modified bond is a phosphorothioate bond).

### (Production Method)

The above-mentioned microRNA according to the present invention and its precursor and derivative can be chemically synthesized by a conventionally known method (synthesis according to a phosphoramidite method using an automatic nucleic acid synthesizer), based on the base sequence thereof. Further, also in regard to a method of introducing a modified structure represented by “-L-M” described above at the 3′-terminal of the oligonucleotide chain that constitutes miRNA produced as such, conventionally known findings (for example, WO 2007/094135, JP 2011-251912 A, and the like) can be referred to as appropriate. Furthermore, also in regard to a method of converting a phosphodiester bond at a particular site to the phosphorus atom-modified structure represented by the chemical formula (3), conventionally known findings can be referred to as appropriate. For example, it is possible to introduce a phosphorothioate bond instead of the phosphodiester bond, by performing oxidation of trivalent phosphorus into pentavalent phosphorus in a final stage of nucleic acid synthesis according to a phosphoramidite method, using a sulfurizing agent solution instead of an oxidizing agent solution.

### [Use]

According to one aspect of the invention, an expression inducer of class I glucose transporter (GLUT) including, as an active ingredient, at least one oligonucleotide selected from the group consisting of hvu-miR168-3p and ata-miR168-5p, their precursors and their derivatives as mentioned above is provided. Here, several classes and molecules have been identified as “class I glucose transporter (GLUT)”. Among the molecules belonging to class I, well known are four types of molecules, GLUT1 to GLUT4, and a target to induce expression by the expression inducer according to the present invention may be any of these, or may be a molecule other than these as long as the molecule belongs to class I. A particularly preferred embodiment of the expression inducer according to the present invention is an expression inducer of GLUT1. A mechanism of the “expression inducer” does not matter as long as the expression inducer is an agent capable of inducing expression of class I GLUT protein.

An effect of inducing expression of class I GLUT by the expression inducer according to the present invention can be confirmed as an index, for example, whether or not the expression level of class I GLUT rises in the presence of the expression inducer as compared with the absence of the agent.

The expression inducer of class I GLUT by the expression inducer according to the present invention includes, as an active ingredient, at least one oligonucleotide selected from the group consisting of hvu-miR168-3p and ata-miR168-5p, their precursors and their derivatives as mentioned above. Here, the phrase “includes, as an active ingredient” means that the expression inducer according to the present invention includes the oligonucleotide in an amount sufficient (that is, an effective amount) for exhibiting a desired expression-inducing activity.

Therefore, the above-mentioned oligonucleotide may be used as it is as an expression inducer, but as long as it includes the active ingredient in such an effective amount and does not impair the expression-inducing activity of class I GLUT, the expression inducer according to the present invention may constitute a composition together with a pharmaceutically acceptable carrier in accordance with a desired product form, other additives, and the like. Moreover, the expression inducer according to the present invention can be mixed with additives such as an excipient and used in a form of a pharmaceutical composition such as a pharmaceutical product and a quasi-drug, that is appropriate for parenteral administration, oral administration or external administration, and a food or beverage composition or the like.

Here, examples of a composition containing the expression inducer of class I GLUT include a pharmaceutical composition for prevention or treatment of a disease or disorder related to glucose metabolism, and a food or beverage composition for prevention or improvement of the disease or disorder. Here, there is no particular limitation on the specific type of the disease or disorder related to glucose metabolism, and conventionally known findings can be referred to as appropriate. An example includes at least one selected from the group consisting of diabetes (hyperglycemia); cardiovascular metabolic syndrome; Alzheimer's disease; Huntington's disease; epilepsy; ischemia; Parkinson's disease; amnesia; dementia; mild cognitive impairment (MCI); attention deficit hyperactivity disorder (ADHD); amyotrophic lateral sclerosis (ALS); and traumatic brain injury. In addition, it is clear that cancer cells undergo energy metabolism biased to glycolysis system that obtains energy faster than efficient oxidative phosphorylation pathway (TCA cycle) and produces a nucleic acid material required for growth (Warburg effect). As a result, energy metabolism of immune cells that exist around the cancer cells and control innate immunity is inhibited, resulting in a state of immunodeficiency, and a more advantageous environment for the cancer cells is created. Here, it is considered that the energy metabolism utilizing the glycolysis system in these cells is restored by allowing the expression inducer according to the present invention to act on such immune cells. Therefore, the above-mentioned “disease or disorder related to glucose metabolism” also includes cancer-induced immunodeficiency.

When provided as a pharmaceutical composition, a therapeutically effective amount of the oligonucleotide is formulated with one or more pharmaceutically acceptable carriers (additives) and/or diluents. As described in detail below, the pharmaceutical composition according to the present invention can be specifically formulated for administration in solid or liquid form. Regarding oral administration, for example, examples include a liquid medicine (aqueous or non-aqueous solution or suspension), a tablet, a bolus, a powder, a granule, and a paste for application to tongue. Regarding parenteral administration, for example, the pharmaceutical composition can be formulated into a preparation for subcutaneous, intraperitoneal or intravenous injection, as a sterile solution or a suspension, or a formulation for topical application, or for intravaginal or intrarectal administration. Among them, the oligonucleotide according to the present invention is preferably formulated in the form of an injection in consideration of the fact that it is an RNA drug.

The “therapeutically effective amount” means an amount of an active material or composition effective to produce any desired therapeutic effect at a reasonable benefit/risk ratio also applicable to any medical treatment. For example, the amount of administration of the expression inducer according to the present invention varies depending on the object disease, the object of administration, the administration route or the like, but since the dose can be easily varied depending on the conditions such as body weight of a person to be treated, the amount of administration can be selected as appropriate by those ordinarily skilled in the art.

The term “pharmaceutically acceptable” is used to refer to a compound, a material, a composition, and/or a dosage form, which is suitable for a proper advantage/risk ratio to the extent of making right medical judgment, and is appropriate for the use of bringing into contact with human and animal tissues without any problems or complications, such as excessive toxicity, stimulation, and allergic reactions.

The “pharmaceutically acceptable carrier” means a pharmaceutically acceptable material, composition or excipient, such as a liquid or solid filler, diluent, plastic remedy, solvent or encapsulation material which are involved in conveyance or transportation of the cell growth inhibitor according to the present invention to one organ or portion of the body to another organ or portion of the body. Each carrier must be compatible with the other components of the formulation and “acceptable” in a sense that it is not harmful to the patient. Some of the materials that can function as pharmaceutically acceptable carriers are illustrated below: sugars such as lactose, glucose, and sucrose; starches such as corn starch and potato starch; cellulose and derivatives thereof, such as carboxymethyl cellulose sodium, ethyl cellulose, and cellulose acetate; powdered tragacanth; malt; gelatin; talc; plastic remedy such as cocoa butter and suppository wax; oils and fats such as peanut oil, cotton seed oil, safflower oil, sesame oil, olive oil, corn oil, and soybean oil; glycols such as propylene glycol; polyols such as glycerin, sorbitol, mannitol, and polyethylene glycol; esters such as ethyl oleate and ethyl laurate; agar-agar; buffering agents such as magnesium hydroxide and aluminum hydroxide; alginic acid; pyrogen-free water; an isotonic sodium chloride solution; Ringer's solution; ethyl alcohol; a phosphate buffer solution; and other non-toxic compatible substances used in drug formulation are included. In some embodiments, the drug preparation is apyrogenic. That is, one that does not raise the body temperature of the patient is preferable.

In addition, wetting agents such as sodium lauryl sulfate and magnesium stearate, emulsifiers and lubricating agents, as well as colorants, releasing agents, coating agents, sweeteners, flavoring agents and flavors, preservatives and antioxidants may also exist in the composition.

Examples of a pharmaceutically acceptable oxidation inhibitor include the following: water-soluble antioxidants such as ascorbic acid, cysteine hydrochloride, sodium hydrogen sulfate, sodium disulfite, and sodium sulfite; oil-soluble antioxidants such as ascorbyl palmitate, butyl hydroxyanisole (BHA), butyl hydroxytoluene (BHT), lecithin, propyl gallate, and α-tocopherol; and metal chelating agents such as citric acid, ethylenediaminetetraacetic acid (EDTA), sorbitol, tartaric acid, and phosphoric acid, can also be contained as necessary.

A pharmaceutical composition containing the expression inducer according to the present invention suitable for parenteral administration contains, together with the oligonucleotide according to the present invention, one or more pharmaceutically acceptable sterile isotonic aqueous solutions or non-aqueous solutions, dispersants, suspensions or emulsions, or sterile powders that can be reconstituted in sterile injectable solutions or dispersants immediately prior to use, which may contain an antioxidant, a buffering agent, a bacteriostat, a solute that makes a preparation isotonic with recipient's blood, or a suspension or a concentrate.

Examples of suitable aqueous and non-aqueous carriers that can be used in the pharmaceutical composition according to the present invention include water, ethanol, polyols (for example, glycerol, propylene glycol, polyethylene glycol, etc.), and suitable mixtures thereof, vegetable oils such as olive oil, as well as injectable organic esters such as ethyl oleate. Inherent fluidity can be maintained, for example, by use of a coating material such as lecithin, by maintenance of required particle size in the case of a dispersant, and by use of a surfactant.

These compositions may include adjuvants such as preservatives, wetting agents, emulsifiers, and dispersants. Prevention of microbial activity can be ensured, for example, by inclusion of various antibacterial agents and antifungal agents such as parabens, chlorobutanol, and phenol sorbate. It is preferable to include an isotonic agent such as sugar or sodium chloride in the composition. Further, sustained absorption of an injectable drug form can be caused by inclusion of an active material that delays absorption, such as aluminum monostearate and gelatin.

In the present invention, the “food or beverage composition” is not particularly limited as long as it is other than a pharmaceutical composition for the purpose of prevention or treatment and is in a form that can be orally ingested by a mammal, and the form thereof also may be any of liquids (solutions, suspensions, emulsions, etc.), semi-liquids, powders, or solid moldings. For this reason, the food or beverage composition may be, for example, in the form of a beverage or in the form of a tablet of a dietary supplement such as a supplement.

Specifically, examples of the form of the food or beverage composition include instant foods such as instant noodles, retort foods, canned foods, microwave foods, instant soups/miso soups, and freeze-dried foods; beverages such as soft drinks, fruit juice drinks, vegetable drinks, soy milk drinks, coffee drinks, tea drinks, powdered drinks, concentrated drinks, nutritional drinks, and alcoholic drinks; flour products such as bread, pasta, noodles, cake mix, flour for fried food, and bread crumb; confectioneries such as candy, caramel, chewing gum, chocolates, cookies, biscuits, cakes, pies, snacks, crackers, Japanese sweets, and desserts; seasonings such as sauces, tomato processing seasonings, flavor seasonings, cooking mixes, gravy, dressings, soups, and curry and stew mixes; oils and fats such as processed fats and oils, butter, margarine, and mayonnaise; dairy products such as milk beverages, yogurts, lactic acid bacteria beverages, ice creams, and creams; processed marine products such as fish hams and sausages and fish paste products; processed livestock products such as meat hams and sausages; processed agricultural products such as canned agricultural products, jams and marmalades, pickles, boiled beans, and cereals; frozen foods; nutritional foods, and the like.

In the food or beverage composition of the present invention, in addition to the above-mentioned active ingredients, an ingredient having another function may be further added. Further, it is possible to provide food or beverage having a function based on other components, in addition to the effect according to the present invention, for example, by blending the active ingredient of the present invention to foods, health foods, functional foods, and supplements (for example, foods containing one or more minerals such as calcium and magnesium, and vitamins such as vitamin K) ingested in daily life.

In the production of the food or beverage composition according to the present invention, sugars, flavors, fruit juices, food additives, stabilizers and the like used in formulation design of ordinary foods and beverages can be appropriately added. The food or beverage composition can be produced with reference to production technology known in the art. The food or beverage composition according to the present invention can take various forms, and the food or beverage composition according to the present invention may be produced according to known production technology for pharmaceutical products. In that case, it can be produced by using the carriers or additives described in the section of production of the expression inducer or pharmaceutical composition according to the present invention. Further, in the production stage, it may be formed as a multifunctional food or beverage composition by combining with other components or other functional foods that exhibit functions other than the functions in the present invention.

When the pharmaceutical composition or food or beverage composition according to the present invention is administered or ingested, the amount of administration or the amount of ingestion of the active ingredient according to the present invention can be determined depending on recipient, age and weight of the recipient, symptoms, administration time, dosage form, administration method, combination of drugs, and the like. In the present invention, it is preferable to appropriately set the content in a pharmaceutical composition or food or beverage composition, in consideration of the amount of administration or the amount of ingestion of the pharmaceutical composition or the food or beverage composition per day so that at least the amount of the active ingredient required for obtaining an effect of inducing expression of class I GLUT can be administered or ingested per day. Meanwhile, the pharmaceutical composition or the food or beverage composition according to the present invention preferably contains the oligonucleotide as an active ingredient in an amount provided in the range of preferably 1 ng to 100 mg, more preferably 10 ng to 10 mg, and further preferably 100 ng to 1 mg per adult per day in terms of the active ingredient.

### EXAMPLES

Hereinafter, examples of the present invention will be described, but the present invention is not limited to these examples.

### Test Example 1

Single-stranded RNA (hvu-miR168-3p) consisting of the following SEQ ID NO: 1 and single-stranded RNA (ata-miR168-5p) consisting of the following SEQ ID NO: 2 were prepared by purchasing from Ambion, respectively. Then, an effect of these single-stranded RNAs on expression of glucose transporter (GLUT) 1 in human skin-derived fibroblasts (ASF-4-1) was evaluated.

(1) SEQ ID NO: 1:
5′-gaucccgccuugcaccaagugaau-3′;
(2) SEQ ID NO: 2:
5′-ucgcuuggugcagaucgggac-3′

ASF-4-1 cells (human skin-derived fibroblasts) were obtained from JCRB Cell Bank (National Institutes of Biomedical Innovation, Health and Nutrition), and the cells were cultured in an incubator (air/CO₂=95/5 (v/v)) at 37° C. using Dalbecco's Modified Eagle's Medium (DMEM).

The ASF-4-1 cells were seeded in a 6-well plate at 0.5 to 0.8×10⁵ cells/mL, and after 24 hours, the cells were transfected with the hvu-miR168-3p or the ata-miR168-5p (at a final concentration of 20 nM or 40 nM), and the culture was further continued. Meanwhile, transfection was carried out by using cationic liposomes (Lipofectamine (registered trademark) RNAiMAX, Life Technologies Corporation) according to the manufacturer's protocol.

### (Western Blotting; ASF-4-1 Cells)

The cells were collected with a cell scraper after 72 hours from the transfection, and the expression level of GLUT1 protein was analyzed by the following Western blotting. Meanwhile, as a negative control plot (Control), a control miRNA purchased from Dharmacon, Inc. was used.

More specifically, the cells were homogenized in an ice-cooled lysis buffer (10 mM Tris-HCl (pH 7.4), 1% (w/v) NP-40, 0.1% (w/v) deoxycholic acid, 0.1% (w/v) SDS, 150 mM NaCl, 1 mM EDTA, and 1% (w/v) Protease Inhibitor Cocktail (Sigma-Aldrich Corporation), and the cells were left to stand on ice for 20 minutes. The homogenate was centrifuged at 13,000 rpm for 20 minutes (4° C.), and then the supernatant was collected as a sample for Western blotting. The protein content in the sample was measured using a DC Protein Assay Kit (manufactured by Bio-Rad Laboratories, Inc.). The sample (protein amount of 5 μg) was separated by SDS-PAGE using a 10.0% or 12.5% (w/v) polyacrylamide gel and was transferred onto a PVDF membrane (Perkin Elmer Life Sciences, Inc.). The sample was incubated in a 5% (w/v) defatted emulsion (prepared with PBS including 0.1% (w/v) Tween (registered trademark) 20 (PBS-T)) for 1 hour, and non-specific bonds were blocked. Thereafter, the membrane was incubated at 4° C. overnight together with a primary antibody (rabbit anti-GLUT1 monoclonal antibody (Cell Signaling Technology, Inc., CST #12939)) that had been adequately diluted with PBS-T including 2% (w/v) bovine serum albumin and 0.01% (w/v) sodium azide. Subsequently, the membrane was washed three times with PBS-T and was further incubated at room temperature together with a secondary antibody (HRP-conjugated anti-rabbit IgG antibody, Cell Signaling Technology, Inc.). Then, the membrane was washed three times with PBS-T. Immunoblotting was visualized using Amersham ECL Plus Western Blotting Detection Reagent (GE Healthcare). β-Actin was used as an internal standard by re-incubating the same membrane using anti-R-Actin antibody (Sigma-Aldrich Corporation).

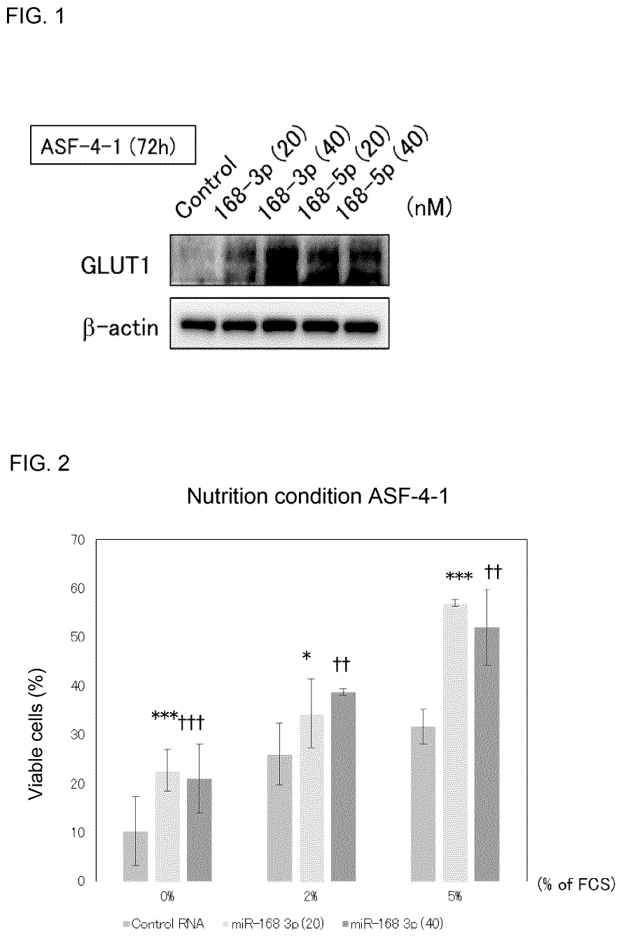

The results of Western blotting are shown in FIG. 1. As shown in FIG. 1, the expression level of GLUT1 protein was markedly increased by transfection with hvu-miR168-3p or ata-miR168-5p in either case at a final concentration of 20 nM and 40 nM.

### Test Example 2

(Effects of miRNA Administration and Nutritional Conditions on Fibroblast Growth)

ASF-4-1 cells were cultured in the same manner as in Test Example 1 described above.

Subsequently, 24 hours after the start of culture, any of control miRNA, hvu-miR-168-3p (at a final concentration of 20 nM) or hvu-miR-168-3p (at a final concentration of 40 nM) was transfected by the same method as above, and the culture was further continued.

Thereafter, 72 hours after the start of culture, for each of the three culture systems described above, 0% (v/v) (without addition), 2% (v/v) or 5% (v/v) fetal calf serum (FCS) was added to the medium to adjust nutritional conditions of the culture system, and the culture was further continued.

Then, 144 hours after the start of culture, the viable cell count was measured by trypan blue staining method. At this time, viable cells (%) were calculated as a relative cell count when the viable cell count in a case where the viable cell count in the negative control plot was designated as 100%. The results are shown in FIG. 2 (* and †: p<0.05, ** and †\: p<0.01, *** and †††: p<0.001; the same applies hereafter). Meanwhile, regarding a statistical analysis, statistical significance was evaluated by a two-tailed Student's t-test using a GraphPad Prism Software system (GraphPad Software, Inc.). Values were shown as mean±standard deviation obtained by evaluating cells from 6 to 12 wells.

As shown in FIG. 2, the viable cell count was significantly increased in either of hvu-miR168-3p and ata-miR168-5p, which are the miRNAs according to the present invention, and under all nutritional conditions, as compared with that of the negative control plot.

### Test Example 3

### (Nude Mouse Sugar Load Test)

Five-week-old male ICR mice (Japan SLC, Inc., Hamamatsu, Shizuoka) were purchased and acclimated to a one-week breeding environment. A miRNA stock solution was prepared by diluting hvu-miR168-3p with a sterile phosphate buffer to 50 μM. This miRNA stock solution was stored at −35° C. until administration. After acclimation to the environment for 1 week, 750 μg/kg (diluted with physiological saline to set the amount of administration to 50 μL) hvu-miR168-3p was administered to each mouse per time from the tail vein of mouse by a lipofection method using lipofectamine RNAi and OPTI-MEM five times every 72 hours. Meanwhile, the same amount of OPTI-MEM containing no miRNA was administered to the negative control plot.

After the end of the administration period, the mice were fasted for 24 hours for the purpose of removing stomach contents for glucose loading. In the glucose load test, a sugar load of 2 g per 1 kg of mouse body weight was performed with a gastric sonde needle, and the blood sugar level was monitored at 30-minute intervals for up to 2 hours after the start of administration. Meanwhile, the blood sugar level was measured by inserting a needle into the tail vein and using a blood sugar level measuring device Medisafe Mini GR102 (Terumo, Shibuya-ku, Tokyo) for blood exuding from the tail vein in a dome shape. A measurement test paper is mounted on a chip attached to a tip of the measuring instrument, and when blood is adsorbed on this chip, glucose in the blood is converted to hydrogen peroxide and gluconic acid by an action of glucose oxidase contained in the test paper. The hydrogen peroxide produced in this way reacts with 4-aminoantipyrine and N-ethyl-N-(2-hydroxy-3-sulfopropyl)-m-toluidine contained in the reaction test paper by an action of peroxidase to produce a quinone-based pigment, and it is possible to measure the blood sugar level by quantifying reddish-purple coloration of this pigment by colorimetric determination.

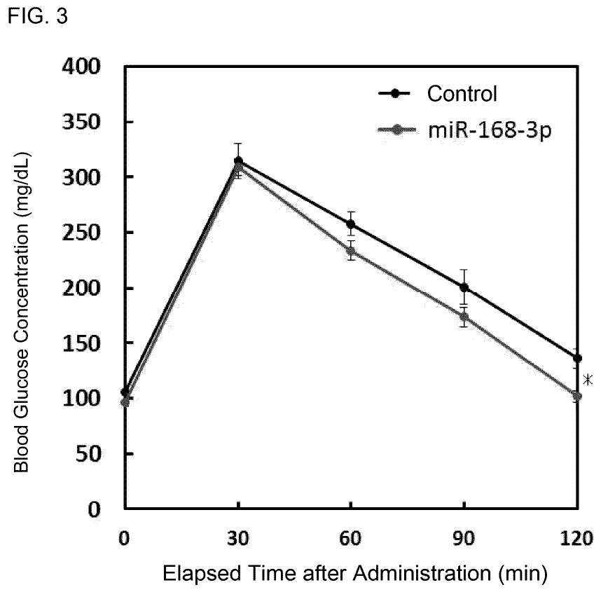

The results are shown in FIG. 3. As shown in FIG. 3, in the mice administered with hvu-miR168-3p, which is the miRNA according to the present invention, the value of blood glucose concentration (blood sugar level) tended to decrease after 60 minutes as compared with the value in the mice in the negative control plot, and a significant difference was seen 120 minutes after the administration (Student's t-test).

### (Western Blotting; Mouse Brain Tissue Homogenate)

Brain tissues of two mice (and two mice in the negative control plot) subjected to the above-mentioned sugar load test were placed in a cell lysis buffer for SDS-PAGE to prepare a homogenate by spatula. Then, the supernatant was separated by subjecting the homogenate to centrifugation treatment, and the expression level of GLUT1 protein was analyzed by the following Western blotting. The protein content in the sample was measured using a DC Protein Assay Kit (manufactured by Bio-Rad Laboratories, Inc.). The sample (protein amount of 5 μg) was separated by SDS-PAGE using a 10.0% or 12.5% (w/v) polyacrylamide gel and was transferred onto a PVDF membrane (Perkin Elmer Life Sciences, Inc.). The sample was incubated in a 5% (w/v) defatted emulsion (prepared with PBS including 0.1% (w/v) Tween (registered trademark) 20 (PBS-T)) for 1 hour, and non-specific bonds were blocked. Thereafter, the membrane was incubated at 4° C. overnight together with a primary antibody (rabbit anti-GLUT1 monoclonal antibody (Cell Signaling Technology, Inc., CST #12939) that had been adequately diluted with PBS-T including 2% (w/v) bovine serum albumin and 0.01% (w/v) sodium azide. Subsequently, the membrane was washed three times with PBS-T and was further incubated at room temperature together with a secondary antibody (HRP-conjugated anti-rabbit IgG antibody, Cell Signaling Technology, Inc.). Then, the membrane was washed three times with PBS-T. Immunoblotting was visualized using Amersham ECL Plus Western Blotting Detection Reagent (GE Healthcare). GAPDH was used as an internal standard by re-incubating the same membrane using anti-GAPDH antibody (Cell Signaling Technology, Inc., CST #2118).

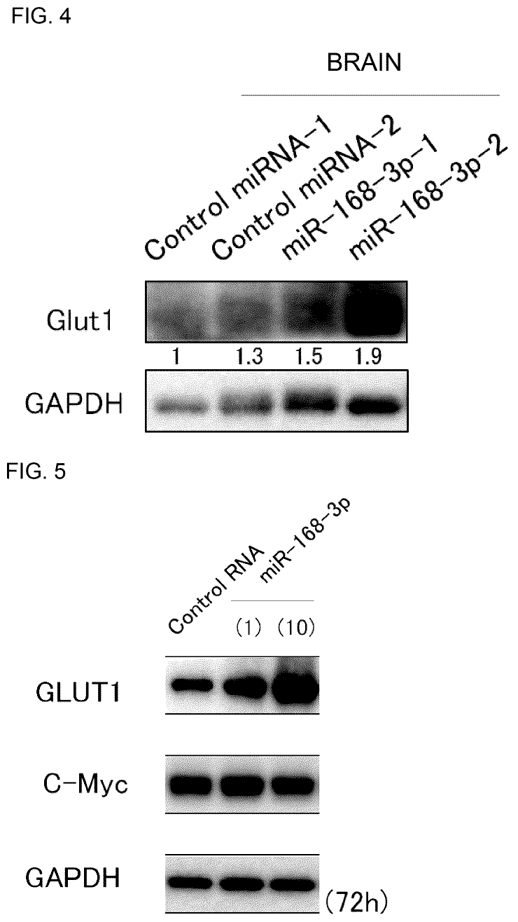

The results of Western blotting are shown in FIG. 4. As shown in FIG. 4, in the brain tissue of the mice administered with hvu-miR168-3p, the expression level of GLUT1 protein was markedly increased as compared with that of the mice in the negative control plot. In addition, when the same Western blotting was performed on a gastrocnemius muscle tissue, it was confirmed that the expression level of GLUT1 protein was significantly increased by administration of hvu-miR168-3p as well.

### Test Example 4

An effect of hvu-miR168-3p on the expression of glucose transporter (GLUT) 1 in human rhabdomyosarcoma-derived cells (JCRB9072) was evaluated.

JCRB9072 cells (human rhabdomyosarcoma-derived cells) were obtained from JCRB Cell Bank (National Institutes of Biomedical Innovation, Health and Nutrition), and the cells were cultured in an incubator (air/CO₂=95/5 (v/v)) at 37° C. using Dalbecco's Modified Eagle's Medium (DMEM).

The JCRB9072 cells were seeded in a 6-well plate at 0.5 to 0.8×10⁵ cells/mL, and after 24 hours, the cells were transfected with the hvu-miR168-3p (at a final concentration of 1 nM or 10 nM), and the culture was further continued. Meanwhile, transfection was carried out by using cationic liposomes (Lipofectamine (registered trademark) RNAiMAX, Life Technologies Corporation) according to the manufacturer's protocol.

### (Western Blotting; JCRB9072 Cells)

The cells were collected after 72 hours from the transfection, the cells were collected with a cell scraper, and the expression level of GLUT1 protein was analyzed by the following Western blotting. Meanwhile, as a negative control plot (Control), a control miRNA purchased from Dharmacon, Inc. was used.

More specifically, the cells were homogenized in an ice-cooled lysis buffer (10 mM Tris-HCl (pH 7.4), 1% (w/v) NP-40, 0.1% (w/v) deoxycholic acid, 0.1% (w/v) SDS, 150 mM NaCl, 1 mM EDTA, and 1% (w/v) Protease Inhibitor Cocktail (Sigma-Aldrich Corporation), and the cells were left to stand on ice for 20 minutes. The homogenate was centrifuged at 13,000 rpm for 20 minutes (4° C.), and then the supernatant was collected as a sample for Western blotting. The protein content in the sample was measured using a DC Protein Assay Kit (manufactured by Bio-Rad Laboratories, Inc.). The sample (protein amount of 5 μg) was separated by SDS-PAGE using a 10.0% or 12.5% (w/v) polyacrylamide gel and was transferred onto a PVDF membrane (Perkin Elmer Life Sciences, Inc.). The sample was incubated in a 5% (w/v) defatted emulsion (prepared with PBS including 0.1% (w/v) Tween (registered trademark) 20 (PBS-T)) for 1 hour, and non-specific bonds were blocked. Thereafter, the membrane was incubated at 4° C. overnight together with a primary antibody (rabbit anti-GLUT1 monoclonal antibody (Cell Signaling Technology, Inc., CST #12939) that had been adequately diluted with PBS-T including 2% (w/v) bovine serum albumin and 0.01% (w/v) sodium azide. Subsequently, the membrane was washed three times with PBS-T and was further incubated at room temperature together with a secondary antibody (HRP-conjugated anti-rabbit IgG antibody, Cell Signaling Technology, Inc.). Then, the membrane was washed three times with PBS-T. Immunoblotting was visualized using Amersham ECL Plus Western Blotting Detection Reagent (GE Healthcare). c-Myc and GAPDH were used as internal standards by re-incubating the same membrane using anti-c-Myc antibody (Cell Signaling Technology, Inc.) and anti-GAPDH antibody (Cell Signaling Technology, Inc.).

The results of Western blotting are shown in FIG. 5. As shown in FIG. 5, the expression level of GLUT1 protein was markedly increased by transfection with hvu-miR168-3p in either case at a final concentration of 1 nM and 10 nM.

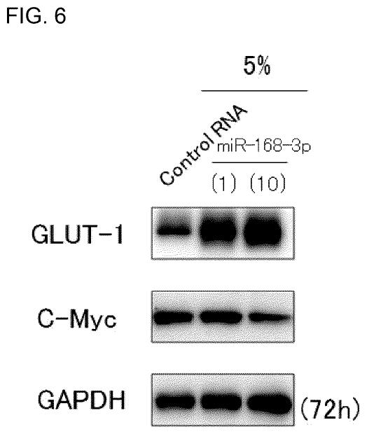

In addition, the results of Western blotting in the same manner as above with addition of 5% (v/v) fetal calf serum (FCS) to a culture medium are shown in FIG. 6. As shown in FIG. 6, the expression level of GLUT1 protein was markedly increased by transfection with hvu-miR168-3p in either case at a final concentration of 1 nM and 10 nM. Moreover, the increase in the expression level of GLUT1 protein was even more remarkable than the results in FIG. 5 in which FCS was not added.

### Test Example 5

(Effects of miRNA Administration and Nutritional Conditions on Sarcoma Cell Growth)

JCRB9072 cells were cultured in the same manner as in Test Example 4 described above.

Subsequently, 24 hours after the start of culture, any of control miRNA, hvu-miR-168-3p (at a final concentration of 1 nM), hvu-miR-168-3p (at a final concentration of 10 nM), ata-miR-168-5p (at a final concentration of 1 nM) or ata-miR-168-5p (at a final concentration of 10 nM) was transfected by the same method as above, and the culture was further continued.

Thereafter, 72 hours after the start of culture, for each of the five culture systems described above, 0% (v/v) (without addition), 2% (v/v) or 5% (v/v) fetal calf serum (FCS) was added to the medium to adjust nutritional conditions of the culture system, and the culture was further continued.

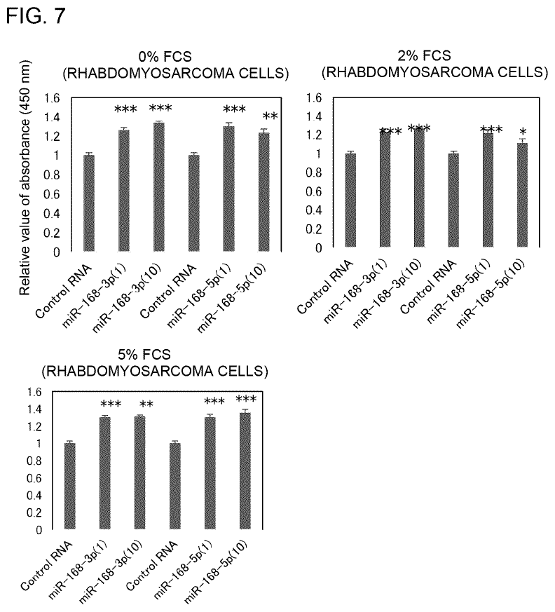

Then, 96 hours after the start of culture, absorbance (450 nm) of the culture system were measured. At this time, the absorbance of each culture system was calculated as a relative value when the absorbance (450 nm) in the negative control plot was designated as 1. The results are shown in FIG. 7 (*: p<0.05, **: p<0.01, ***: p<0.001). Meanwhile, regarding a statistical analysis, statistical significance was evaluated by a two-tailed Student's t-test using a GraphPad Prism Software system (GraphPad Software, Inc.). Values were shown as mean±standard deviation obtained by evaluating cells from 6 to 12 wells.

As shown in FIG. 7, the absorbance was significantly increased in either of hvu-miR168-3p and ata-miR168-5p, which are the miRNAs according to the present invention, and under all nutritional conditions, as compared with that of the negative control plot.

(Effect of miRNA Administration on ATP Levels in Sarcoma Cells)

For the samples whose absorbance was measured above, the culture system in which the amount of FCS added was 5% (v/v), intracellular ATP levels were measured using an ATP measurement kit (A22066 manufactured by Thermo Fisher Scientific).

Specifically, first, a reaction reagent was prepared according to the protocol attached to the kit and stored in a dark place. At this time, after the addition of firefly luciferase, the reagent was not vibrated and was gently inverted and mixed.

Meanwhile, the cultured cells were washed with PBS buffer and the cells were detached from the plate by trypsin treatment (trypsin was added to each well of the 6-well plate at 500 μL/well). Thereafter, the cells diluted with addition of an equal amount of medium were collected in a new Eppendorf tube. Then, 10 μL each of the collected sample was gently dispensed into bottom of three separate Eppendorf tubes. At this time, it is preferable that the cells are separated as much as possible, but care was taken not to give vibration because the ATP in the cells would elute due to destruction of the cells.

Subsequently, 90 μL each of the reaction reagent prepared above was dispensed into the sample in each tube. Then, each sample was lightly spun down and reacted in a dark place at room temperature for 15 minutes. During this reaction, concentration of cells collected above was counted.

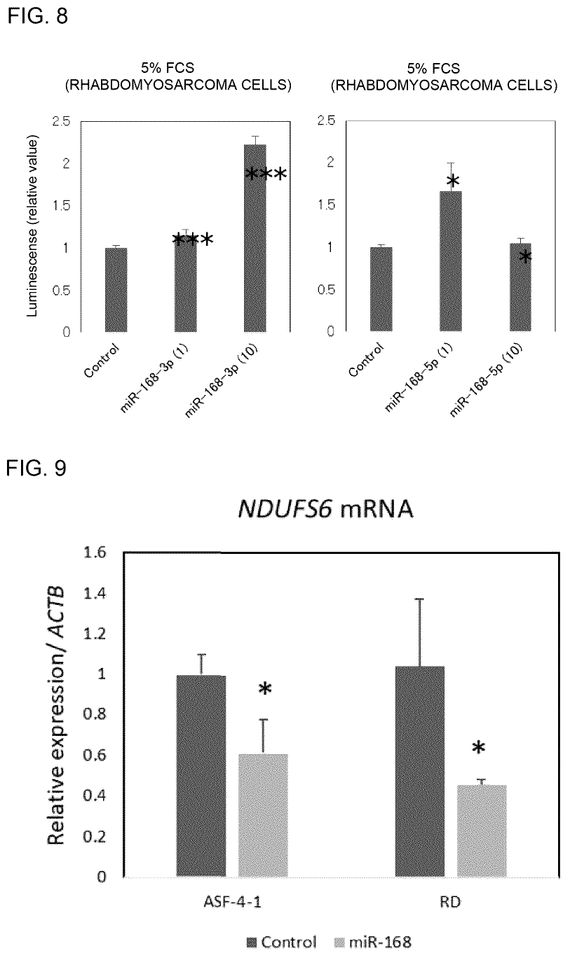

After the completion of the reaction, luciferase activity of each sample was measured according to the protocol of the kit, and the ATP level per cell was calculated using the counted number of cells. At this time, the luminescence amount of each culture system was calculated as a relative value when the luminescence amount in the negative control plot was designated as 1. The results are shown in FIG. 8. (*: p<0.05, ***: p<0.001). Meanwhile, regarding a statistical analysis, statistical significance was evaluated by a two-tailed Student's t-test using a GraphPad Prism Software system (GraphPad Software, Inc.). Values were shown as mean±standard deviation obtained by evaluating cells from 6 to 12 wells.

As shown in FIG. 8, the intracellular ATP levels in the rhabdomyosarcoma cells cultured with 5% (v/v) FCS added were significantly increased in either of hvu-miR168-3p and ata-miR168-5p, which are the miRNAs according to the present invention. In particular, the increase in ATP level was remarkable when hvu-miR168-3p was administered.

### Test Example 6

(Effect of miRNA Introduction on Gene Expression (Microarray Analysis))

For the purpose of evaluating an effect of introduction of hvu-miR168-3p on gene (mRNA) expression in human skin-derived fibroblasts (ASF-4-1 cells) and human rhabdomyosarcoma-derived cells (JCRB9072 cells), microarray analysis was performed on genes belonging to SCL2 (GLUT) family. The results are shown in Table 1 below. Meanwhile, the larger the numerical value of “FC ASF” shown in Table 1, the more enhanced the expression in the ASF-4-1 cells, and the larger the numerical value of “FC RD”, the more enhanced the expression in the JCRB9072 cells.

TABLE 1

| | GENE | | | |
|---|---|---|---|---|
| ID | SYMBOL | GENE NAME | FC ASF | FC RD |
| A_23_P571 | SLC2A1 | solute carrier family 2 (facilitated glucose transporter) | 1.087164 | 2.889548 |
| A_22_P00023942 | SLC2A1-AS1 | SLC2A1 antisense RNA 1 | 1.814059 | 3.130862 |
| A_33_P3694746 | SLC2A1-AS1 | SLC2A1 antisense RNA 1 | 1.931661 | 2.589347 |
| A_24_P271323 | SLC2A10 | solute carrier family 2 (facilitated glucose transporter) | 1.05114 | 1.228021 |
| A_23_P404565 | SLC2A11 | solute carrier family 2 (facilitated glucose transporter) | 1.607622 | 1.482097 |
| A_33_P6549164 | SLC2A11 | solute carrier family 2 (facilitated glucose transporter) | 1.100015 | 0.94413 |
| A_33_P3268649 | SLC2A11 | solute carrier family 2 (facilitated glucose transporter) | 0.873111 | 0.922595 |
| A_33_P3331125 | SLC2A12 | solute carrier family 2 (facilitated glucose transporter) | 1.048599 | 1.734275 |
| A_22_P00024298 | SLC2A13 | solute carrier family 2 (facilitated glucose transporter) | 1.062179 | 1.135762 |
| A 24_P924816 | SLC2A13 | solute carrier family 2 (facilitated glucose transporter) | 0.718061 | 0.995912 |
| A 33_P3354514 | SLC2A13 | solute carrier family 2 (facilitated glucose transporter) | 0.853957 | 0.930858 |
| A_32_P47754 | SLC2A14 | solute carrier family 2 (facilitated glucose transporter) | 0.566062 | 1.08416 |
| A_24_P405705 | SLC2A2 | solute carrier family 2 (facilitated glucose transporter) | 2.400556 | 0.916188 |
| A_23_P139669 | SLC2A3 | solute carrier family 2 (facilitated glucose transporter) | 0.704645 | 1.362202 |
| A_24_P81900 | SLC2A3 | solute carrier family 2 (facilitated glucose transporter) | 0.586186 | 1.31369 |
| A_32_P151263 | SLC2A4 | solute carrier family 2 (facilitated glucose transporter) | 0.306221 | 0.718176 |
| A_23_P102571 | SLC2A4RG | SLC2A4 regulator | 1.067245 | 0.88747 |
| A_33_P3317797 | SLC2A5 | (solute carrier family 2 (facilitated glucose/fructose transporter) | 0.963947 | 1.121762 |
| A_24_P111054 | SLC2A5 | solute carrier family 2 (facilitated glucose/fructose transporter) | 0.863851 | 0.883549 |
| A_23_P160159 | SLC2A5 | solute carrier family 2 (facilitated glucose/fructose transporter) | 1.072699 | 0.841104 |
| A_33_P3398912 | SLC2A6 | solute carrier family 2 (facilitated glucose transporter) | 0.682242 | 1.080883 |
| A_33_P3337044 | SLC2A6 | solute carrier family 2 (facilitated glucose transporter) | 1.241748 | 1.078532 |
| A_33_P3238425 | SLC2A7 | solute carrier family 2 (facilitated glucose transporter) | 0.960235 | 0.916188 |
| A_33_P3418158 | SLC2A8 | solute carrier family 2 (facilitated glucose transporter) | 0.517002 | 0.855413 |
| A_23_P252783 | SLC2A8 | solute carrier family 2 (facilitated glucose transporter) | 0.731699 | 0.724398 |
| A_23_P213014 | SLC2A9 | solute carrier family 2 (facilitated glucose transporter) | 0.760872 | 0.916188 |

As shown in Table 1, it can be seen that, by the introduction of hvu-miR168-3p, the expressions of SLC2A1 gene, which corresponds to the systemically expressed GLUT1 gene, and the SLC2A2 gene, which corresponds to GLUT2 gene and specifically expressed in the small intestinal epithelium, were particularly enhanced. In addition, also for other genes belonging to the SLC2 (GLUT) family, enhanced expression of some genes was observed.

### Test Example 7

In general, it is known that a microRNA has various functions by controlling a plurality of target genes. Therefore, signal transduction pathways affected by the introduction of hvu-miR168-3p were investigated by KEGG Pathway analysis, and pathways in which gene expression was both increased or both reduced in both ASF-4-1 cells and JCRB9072 cells were investigated. Among the results, pathways in which gene expression was both increased are shown in Table 2 below.

TABLE 2
Pathways with increased expression by miR-168 treatment

| NAME | NOM p-val | FDR q-val | FWERp-val |
|---|---|---|---|
| KEGG_GRAFT_VERSUS_HOST_DISEASE | 0 | 0.02196083 | 0.019 |
| KEGG_NOD_LIKE_RECEPTOR_SIGN ALING_PATH WAY | 0 | 0.02972656 | 0.052 |
| KEGG_LEISHMANIA_INFECTION | 0 | 0.06904901 | 0.266 |
| KEGG_CYTOKINE_CYTOKINE_RECEPTOR_INTERACTION | 0.00215054 | 0.21366394 | 0.899 |
| KEGG_ALLOGRAFT_REJECTION | 0.00457666 | 0.08692295 | 0.205 |
| KEGG_AUTOIMMUNE_THYROID_DISEASE | 0.00469484 | 0.08236409 | 0.254 |
| KEGG_ASTHMA | 0.01106195 | 0.09858763 | 0.411 |
| KEGG_EPITHELIAL_CELL_SIGNALING_IN_HELICOBACTER_PYLORI_INFECTION | 0.01573034 | 0.22761495 | 0.893 |
| KEGG_TOLL_LIKE_RECEPTOR_SIGNALING_PATHWAY | 0.01709402 | 0.22502196 | 0.922 |
| KEGG_LEUKOCYTE_TRANSENDOTHELIAL_MIGRATION | 0.01978022 | 0.23199637 | 0.874 |
| KEGG_PROXIMAL_TUBULE_BICARBONATE_RECLAMATION | 0.0212766 | 0.15474482 | 0.624 |
| KEGG_NITROGEN_METABOLISM | 0.02985075 | 0.14249454 | 0.648 |
| KEGG_ANTIGEN_PROCESSING_AND_PRESENTATION | 0.04347826 | 0.24720389 | 0.947 |
| KEGG_GLYCOSPHINGOLIPID_BIOSYNTHESIS_LACTOAND_NEOLACTO_SERIES | 0.04411765 | 0.15721901 | 0.709 |

As shown in Table 2, it can be seen that most of the signal transduction pathways in which gene expression was increased by the introduction of hvu-miR168-3p were pathways involved in a reaction to foreign substances (innate immunity) in the living body.

On the other hand, among the results of KEGG Pathway analysis, pathways in which gene expression was both reduced are shown in Table 3 below.

TABLE 3
Pathways with reduced expression by miR-168 treatment

| NAME | NOM p-val | FDR q-val | FWERp-val |
|---|---|---|---|
| KEGG_OXIDATIVE_PHOSPHORYLATION | 0 | 0.14469561 | 0.143 |
| KEGG_PARKINSONS_DISEASE | 0 | 0.06840264 | 0.198 |
| KEGG_RIBOSOME | 0.0018622 | 0.0607844 | 0.229 |
| KEGG_O_GLYCAN_BIOSYNTHESIS | 0.00383877 | 0.08931185 | 0.171 |
| KEGG_HUNTINGTONS_DISEASE | 0.02640845 | 0.5687599 | 0.991 |
| KEGG_SPLICEOSOME | 0.03014184 | 0.75448555 | 0.979 |

As shown in Table 3, signal transduction pathways in which gene expression was reduced by the introduction of hvu-miR168-3p included oxidative phosphorylation (KEGG_OXIDATIVE_PHOSPHORYLATION) pathway.

Based on the above results, among genes located in the oxidative phosphorylation (KEGG_OXIDATIVE_PHOSPHORYLATION) pathway, genes whose expression was reduced by the introduction of hvu-miR168-3p were extracted. The results are shown in Table 4 below.

TABLE 4

| ASF | RD | ASF | RD | | | |
|---|---|---|---|---|---|---|
| CTL | CTL | 168 | 168 | SampleName | | |
| R | I | C | P | ATP6V1G3 | ATP6V1G3 | ATPase, H+ transporting, lysosmal 13 kDa, V1 subunit G3 |
| R | C | I | P | NDUFS6 | NDUFS6 | NADH dehydrogenase (ubiquinone) Fe—S protein 6, 13 kDa |
| | | | | | | (NADH-coenzyme 0 reductase) |
| B | R | I | P | COX6B2 | COX6B2 | cytochrome c oxidase subunit VIb polypeptide 2 (testis) |
| O | R | P | B | NDUFB7 | NDUFB7 | NADH dehydrogenase (ubiquinone) 1 beta subcomplex, 7, 18 kDa |
| K | R | P | V | NDUFB8 | NDUFB8 | NADH dehydrogenase (ubiquinone) 1 beta subcomplex, 8, 19 kDa |
| R | R | P | P | NDUFA11 | NDUFA11 | NADH dehydrogenase (ubiquinone) 1 alpha subcomplex, 11, 14, 7 kDa |
| L | R | I | P | NDUFC2 | NDUFC2 | NADH dehydrogenase (ubiquinone)1, subcomplex unknown, 2, 14, 5 kDa |
| O | R | P | O | NDUFV3 | NDUFV3 | NADH dehydrogenase (ubiquinone) flavoprotein 3, 10 kDa |
| R | L | C | P | LHPP | LHPP | — |
| R | P | C | P | COX7A1 | COX7A1 | cytochrome c oxidase subunit VIIa polypeptide 1 (muscle) |
| V | R | P | V | NDUFA9 | NDUFA9 | NADH dehydrogenase (ubiquinone) 1 alpha subcomplex, 9, 39 kDa |
| K | R | P | K | NDUFS5 | NDUFS5 | NADH dehydrogenase (ubiquinone) Fe—S protein 5, 15 kDa |
| | | | | | | (NADH-coenzyme 0 reductase) |
| V | R | P | K | COX6B1 | COX6B1 | cytochrome c oxidase subunit Vib polypeptide 1 (ubiquitous) |

As shown in Table 4, the genes involved in oxidative phosphorylation whose expression was reduced by the introduction of hvu-miR168-3p were mainly ATPase, NADH dehydrogenase, and cytochrome c oxidase. From this, it is considered that the induction (enhancement) of the expression of class I GLUT gene by administration of hvu-miR168-3p occurred by a compensatory activation of glycolysis system in order to compensate for decrease in function of oxidative phosphorylation pathway (TCA cycle).

### Test Example 8

(Effect of miRNA Introduction on Gene Expression in Oxidative Phosphorylation Pathway)

Based on the above results, changes in mRNA expression levels of Ndufs6 (NADH dehydrogenase [ubiquinone] iron-sulfur protein 6, mitochondrial), which is a gene involved in oxidative phosphorylation pathway (OXPHOS) in mitochondrial complex I, in ASF-4-1 cells and JCRB9072 cells into which hvu-miR168-3p was introduced were measured by real-time PCR method. Meanwhile, an actin gene was used as an internal standard. The results are shown in FIG. 9.

As shown in FIG. 9, it can be seen that the mRNA expression levels of the Ndufs6 gene were significantly reduced by the introduction of hvu-miR168-3p in either cells. This supports a hypothesis regarding a mechanism of induction (enhancement) of the expression of class I GLUT gene by administration of the miRNA according to the present invention as described above.

This application is based on Japanese Patent Application No. 2018-193615 filed on Oct. 12, 2018, the disclosure of which is incorporated by reference in its entirety.

### [SEQ ID NO: 1]

It is an RNA sequence of hvu-miR168-3p, which is one of the oligonucleotides according to the present invention.

### [SEQ ID NO: 2]

It is an RNA sequence of ata-miR168-5p, which is one of the oligonucleotides according to the present invention.

1. A method for inducing an expression of class I glucose transporter (GLUT) comprising administering to a subject at least one oligonucleotide selected from the group consisting of hvu-miR168-3p and ata-miR168-5p, their precursors and their derivatives.

2. The method according to claim 1, wherein the active ingredient is a single-stranded RNA consisting of a base sequence shown in SEQ ID NO: 1 or SEQ ID NO: 2 below:
(1) SEQ ID NO: 1:
5′-gauccegccuugcaccaagugaau-3′;
(2) SEQ ID NO: 2:
5′-ucgcuuggugcagaucgggac-3′.

3. The method according to claim 1, wherein the derivative has a 3′-terminal modified structure represented by the following chemical formula 1:
-L-M-3′ (1)
in the chemical formula 1, L represents a substituted or unsubstituted cyclic compound-containing group represented by any of the following chemical formulas (2a) to (2g): 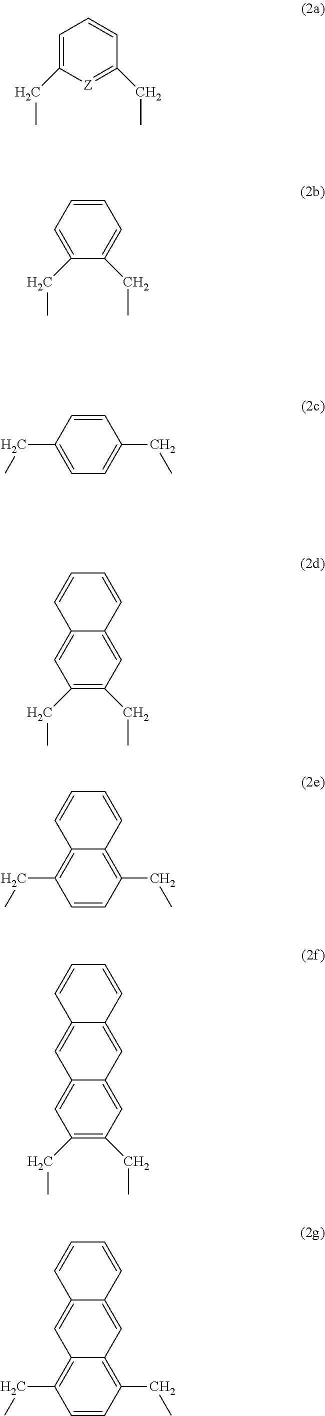
wherein, in the chemical formula (2a), Z represents CH or N,
or L represents a divalent group in which two or more of the cyclic compound-containing groups are respectively linked through a phosphodiester bond; and M represents a hydrogen atom or a hydroxyl protecting group.

4. The method according to claim 1, wherein,
in the derivative, the nucleotides constituting the oligonucleotide are each independently substituted by a base containing a modified sugar moiety selected from the group consisting of 2′-O-methyl, 2′-methoxyethoxy, 2′-fluoro, 2′-allyl, 2′-O-[2-(methylamino)-2-oxoethyl], 4′-thio, 4′-(CH₂)₂—O-2′-crosslinking, 2′-locked nucleic acid and 2′-O—(N-methylcarbamate), and/or the phosphodiester bonds constituting the oligonucleotide are each independently substituted by a phosphorus atom-modified bond represented by the following chemical formula (4): 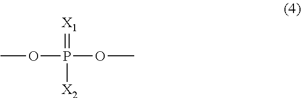
wherein, in the chemical formula (4), X¹s independently represent 0, S or Se, and X²s independently represent OH or O⁻, SH or S⁻, SeH or Se⁻, an alkyl group having 1 to 4 carbon atoms or a morpholino group (provided that the case where X¹ is O and X² is O⁻ is excluded).

5. The method according to claim 1, wherein the class I glucose transporter (GLUT) is glucose transporter (GLUT) 1.

6. A pharmaceutical composition comprising at least one oligonucleotide selected from the group consisting of hvu-miR168-3p and ata-miR168-5p, their precursors and their derivatives, and a pharmaceutically acceptable carrier.

7. A method for preventing or treating a disease or disorder related to glucose metabolism comprising administering to a subject at least one oligonucleotide selected from the group consisting of hvu-miR168-3p and ata-miR168-5p, their precursors and their derivatives.

8. The method according to claim 7, wherein the disease or disorder related to glucose metabolism is at least one selected from the group consisting of diabetes (hyperglycemia); cardiovascular metabolic syndrome; Alzheimer's disease; Huntington's disease; epilepsy; ischemia; Parkinson's disease; amnesia; dementia; mild cognitive impairment (MCI); attention deficit hyperactivity disorder (ADHD); amyotrophic lateral sclerosis (ALS); traumatic brain injury; and cancer-induced immunodeficiency.

9. The pharmaceutical composition according to claim 6, wherein the oligonucleotide is contained in an expression vector expressing the oligonucleotide.

10. A food or beverage composition comprising at least one oligonucleotide selected from the group consisting of hvu-miR168-3p and ata-miR168-5p, their precursors and their derivatives.

11. The food or beverage composition according to claim 10, which is for prevention or improvement of a disease or disorder related to glucose metabolism.

12. The food or beverage composition according to claim 11, wherein the disease or disorder related to glucose metabolism is at least one selected from the group consisting of diabetes (hyperglycemia); cardiovascular metabolic syndrome; Alzheimer's disease; Huntington's disease; epilepsy; ischemia; Parkinson's disease; amnesia; dementia; mild cognitive impairment (MCI); attention deficit hyperactivity disorder (ADHD); amyotrophic lateral sclerosis (ALS); traumatic brain injury; and cancer-induced immunodeficiency.

13. The food or beverage composition according to claim 10, wherein the oligonucleotide is contained in an expression vector expressing the oligonucleotide.
